(19) United States
(12) Patent Application Publication
(10) Pub. No.: US 2022/0289703 A1
(43) Pub. Date: Sep. 15, 2022

(54) METHODS FOR CONVERTING TETRAHYDROCANNABINOLIC ACID INTO CANNABINOLIC ACID

(71) Applicant: Canopy Growth Corporation, Smiths Falls (CA)

(72) Inventors: Christopher ADAIR, Smiths Falls (CA); Mahmood AZIZPOOR FARD, Smiths Falls (CA); Ben GEILING, Smiths Falls (CA)

(21) Appl. No.: 17/637,291

(22) PCT Filed: Aug. 21, 2020
(86) PCT No.: PCT/CA2020/051148

§ 371 Date: Feb. 22, 2022

Related U.S. Application Data
(60) Provisional application No. 62/891,015, filed on Aug. 23, 2019.

(51) Int. Cl. C07D 311/80; A61K 36/185; A61K 31/352

(52) U.S. Cl. CPC C07D 311/80; A61K 36/185; A61K 31/352

(57) ABSTRACT

Disclosed herein is a method for converting tetrahydrocannabinolic acid (THCA) to cannabinolic acid (CBNA). The method comprises contacting an input material comprising THCA with a benzoquinone reagent under reaction conditions comprising: (i) a reaction temperature that is within a target reaction-temperature range; and (ii) a reaction time that is within a target reaction-time range, to provide an output material in which at least a portion of the THCA from the input material has been converted into CBNA.

### CROSS-REFERENCE TO RELATED APPLICATIONS

This application claims priority to and benefit of U.S. Provisional Patent Application Ser. No. 62/891,015 filed on Aug. 23, 2019, which is hereby incorporated by reference in its entirety.

### TECHNICAL FIELD

The present disclosure generally relates to preparing acid-form cannabinoids. In particular, the present disclosure relates to methods for converting tetrahydrocannabinolic acid (THCA) into cannabinolic acid (CBNA).

### BACKGROUND

Cannabinoids are a diverse class of compounds that may be characterized in pharmacological terms, chemical-terms, and/or based on their origin. Many cannabinoids are derived from natural sources, and naturally-derived cannabinoids are often present their acidic forms. The process of converting an acid-form cannabinoid to its neutral form is known as “decarboxylation”, and it is largely associated with the “activation” of the psychoactive effects of a number of cannabinoids. As a chemical transformation, decarboxylation is enthalpically and entropically favourable—it converts a carboxylic-acid-substituted cannabinoid to a neutral cannabinoid and CO₂ (g)—and mild heating is often sufficient to induce decarboxylation.

Given that the energy barriers to cannabinoid decarboxylation are relatively low, methods for converting acid-form cannabinoids into other acid-form cannabinoids (i.e. converting a first cannabinoid into a second cannabinoid without sacrificing the carboxylic-acid functionality) are uncommon. At the same time, research into the medicinal and recreational applications of acid-form cannabinoids is uncovering a number of benefits—both as pure compounds and as components in broad-spectrum cannabinoid mixtures. Moreover, acid-form cannabinoids are of interest to organic chemists as potential synthons for fine-chemical development. Accordingly, methods for preparing acidic-form cannabinoids are desirable. In particular, there is an unmet need for methods of converting acid-form cannabinoids that are naturally abundant into ones that are less so.

### SUMMARY

Tetrahydrocannabinolic acid (THCA) is a well-known cannabinoid that is naturally abundant in a variety of cannabis cultivars. As such, it is potentially useful as a feedstock for the synthesis of less naturally abundant cannabinoids. Cannabinolic acid (CBNA) is one such cannabinoid, and it is a useful target, for example because as research into the potential medical and recreational applications of CBNA is in its infancy. As indicated by the examples and teachings set out herein, the present disclosure provides methods for converting THCA into CBNA. Importantly, the methods of the present disclosure can be tuned to minimize the formation of decaryboxylated by-products—namely THC and CBN. Further, the methods of the present disclosure can be configured to: (i) employ simple, cost-effective techniques; (ii) avoid the use of harmful solvents, such as benzene; and (iii) not require high-purity input material.

In select embodiments, the present disclosure relates to a method for converting THCA into CBNA, the method comprising contacting an input material comprising THCA with a benzoquinone reagent under reaction conditions comprising: (i) a reaction temperature that is within a target reaction-temperature range; and (ii) a reaction time that is within a target reaction-time range, to provide an output material in which at least a portion of the THCA from the input material has been converted into CBNA.

In select embodiments, the present disclosure relates to a method for converting THCA into CBNA, the method comprising contacting the THCA with tetrachloro-1,4-benzoquinone under reaction conditions comprising: (i) a reaction temperature of between about 60° C. and about 130° C.; and (ii) a reaction time that is between about 1 h and about 100 h, such that at least a portion of the THCA is oxidized to CBNA.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a high-performance liquid chromatography with diode-array detection (HPLC-DAD) chromatogram of an output material obtained from a first method in accordance with the present disclosure.

FIG. 2 shows an HPLC-DAD chromatogram of an output material obtained from a second method in accordance with the present disclosure.

FIG. 3 shows an HPLC-DAD chromatogram of an output material obtained from a third method in accordance with the present disclosure.

FIG. 4 shows HPLC-DAD chromatograms of the output material obtained by a method in accordance with the present disclosure (FIG. 4A), the material obtained by purification by chromatography (FIG. 4B), and CBNA crystals obtained from the purified material (FIG. 4C).

FIG. 5 shows an ORTEP drawing of CBNA produced by a method in accordance with the present disclosure.

### DETAILED DESCRIPTION

As noted above, Tetrahydrocannabinolic acid (THCA) is potentially useful as a feedstock for the synthesis of less naturally abundant cannabinoids, for example because it is naturally abundant in a variety of cannabis cultivars. As indicated by the examples and teachings set out herein, the present disclosure provides methods for converting THCA into cannabinolic acid (CBNA). Importantly, the methods of the present disclosure can be tuned to minimize the formation of decaryboxylated by-products—namely THC and CBN. In this respect, experiments towards the methods of the present disclosure indicate that solvent, reaction temperature, and reaction time may influence the extent to which decarboxylation occurs. The methods of the present disclosure may be configured to employ simple, cost-effective techniques. In this respect, experiments towards the methods of the present disclosure indicate that reactions configured to consume substantially all the THCA in the input material may enable purification without chromatography. The methods of the present disclosure may also be configured to avoid the use of harmful solvents, such as benzene. For example, experiments towards the methods of the present disclosure indicate that Class III solvents (such as heptane and ethyl acetate) may be utilized in the conversion of THCA to CBNA. The methods of the present disclosure may also be configured to not require high-purity input material.

In select embodiments, the present disclosure relates to a method for converting THCA into CBNA, the method comprising contacting an input material comprising the THCA with a benzoquinone reagent under reaction conditions comprising: (i) a reaction temperature that is within a target reaction-temperature range; and (ii) a reaction time that is within a target reaction-time range, to provide an output material in which at least a portion of the THCA from the input material has been converted into CBNA.

In select embodiments, the present disclosure relates to a method for converting THCA into CBNA, the method comprising contacting the THCA with tetrachloro-1,4-benzoquinone under reaction conditions comprising: (i) a reaction temperature of between about 60° C. and about 130° C.; and (ii) a reaction time that is between about 1 h and about 100 h, such that at least a portion of the THCA is oxidized to CBNA.

In the context of the present disclosure, the term “contacting” and its derivatives is intended to refer to bringing the input material comprising the THCA and the benzoquinone reagent as disclosed herein into proximity such that a chemical reaction can occur. In some embodiments of the present disclosure, the contacting may be by adding the benzoquinone reagent to the input material comprising THCA. In some embodiments, the contacting may be by combining, mixing, or both.

In select embodiments of the present disclosure, the input material may be a complex mixture of cannabinoids. In the context of the present disclosure, a “complex cannabinoid mixture” is any compositions that comprises at least two cannabinoids, and a “broad-spectrum cannabinoid composition” is one that contains at least three cannabinoids. In the context of the present disclosure, both complex cannabinoid mixtures, and broad-spectrum cannabinoid compositions may further comprise non-cannabinoid compounds such as waxes, oils, terpenes, and the like.

As used herein, the term “cannabinoid” refers to: (i) a chemical compound belonging to a class of secondary compounds commonly found in plants of genus cannabis, (ii) synthetic cannabinoids and any enantiomers thereof; and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11 nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

As used herein, the term “THC” refers to tetrahydrocannabinol. “THC” is used interchangeably herein with “Δ9-THC”. As used herein, the term “THCA” refers to tetrahydrocannabinolic acid.

In select embodiments of the present disclosure, input material may comprise THCA (Δ9-THCA), Δ8-THCA, trans-Δ10-THCA, cis-Δ10-THCA, THCV-A (Δ9-THCV-A), Δ8-THCVA, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran

Structural formulae of cannabinoids of the present disclosure may include the following:

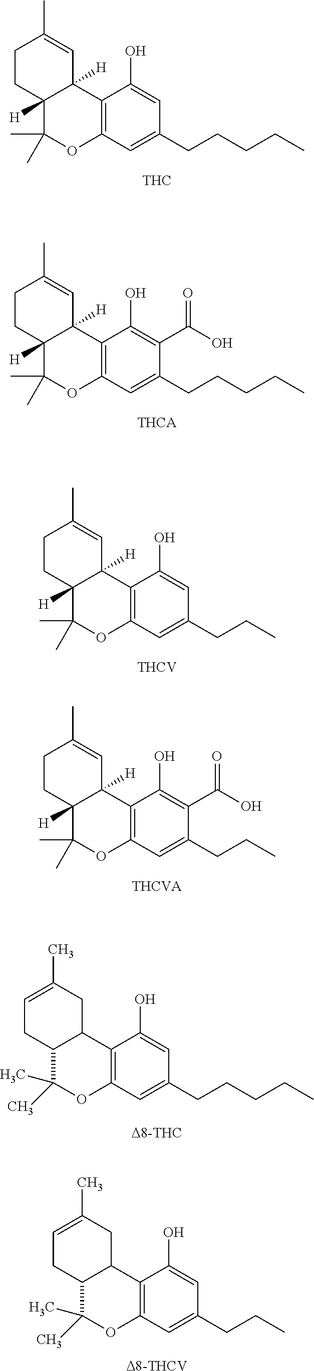

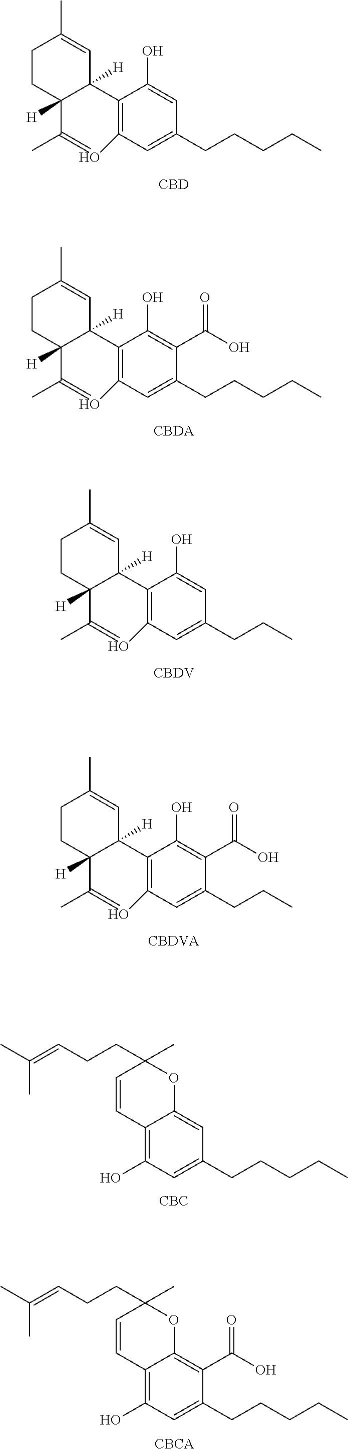

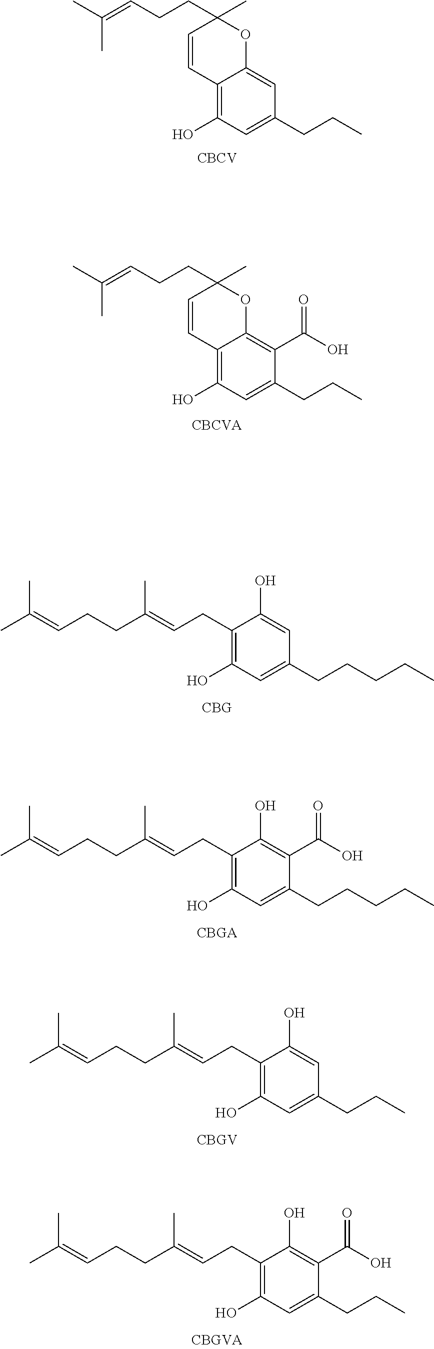

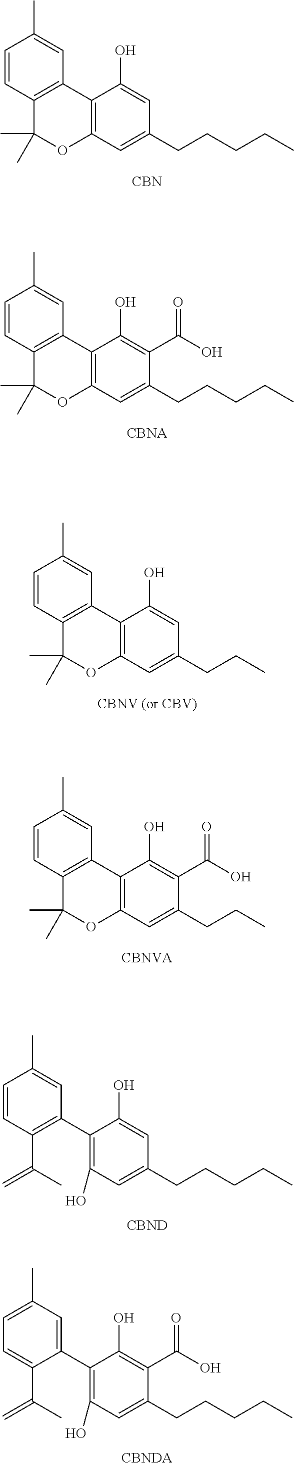

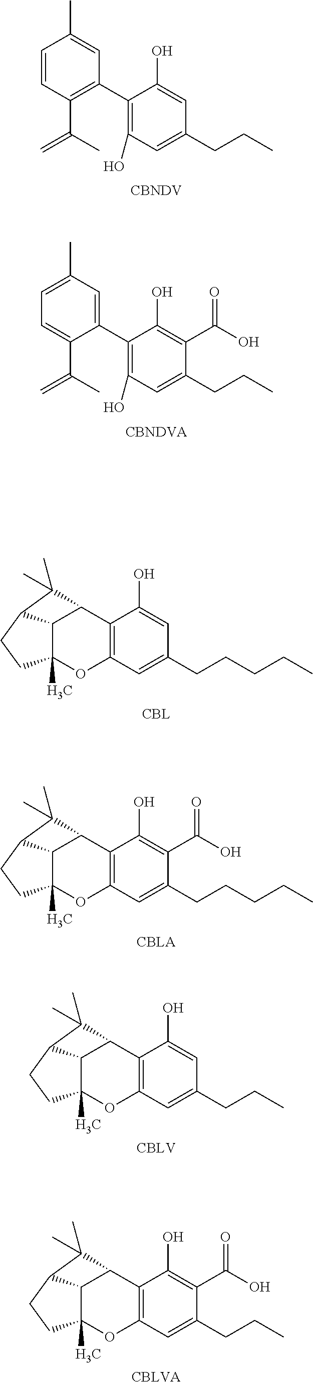

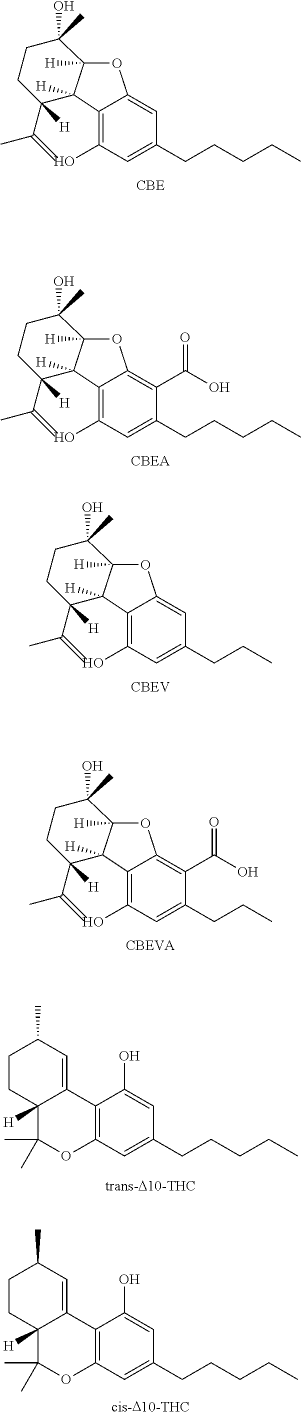

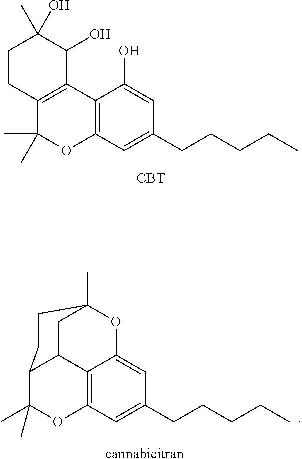

In select embodiments of the present disclosure, the input material may be derived from marijuana biomass. In select embodiments of the present disclosure, the input material may be a distillate, a resin, an extract, or a combination thereof. In select embodiments of the present disclosure, the input material may be THCA in an isolated form. In the context of the present disclosure, an input material that is comprised of a cannabinoid in its “isolated” form is one that does not contain substantial quantities of other cannabinoids and/or non-cannabinoid compounds. For example, an input material having a cannabinoid in its isolated form may be comprised of at least about 95 wt. %, at least about 99 wt. %, or at least about 99.5 wt. % of that cannabinoid. Those skilled in the art who have benefitted from the teachings of the present disclosure will recognize that such isolated forms may include trace amounts of other cannabinoid and/or non-cannabinoid compounds.

In select embodiments of the present disclosure, at least about 80 wt. % of the THCA in the input material may be Δ⁹-THCA. In select embodiments of the present disclosure, at least about 95 wt. % of the THCA may be Δ⁹-THCA.

In the context of the present disclosure, the relative quantities of cannabinoids in a mixture may be expressed as a ratio such as CBNA:non-CBNA cannabinoid. Those skilled in the art will recognize that a variety of analytical methods may be used to determine such ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof

In select embodiments of the present disclosure, the output material may further comprises cannabinol (CBN), and the output material may have a CBNA:CBN ratio of at least about 5.0:1.0. In select embodiments of the present disclosure, the CBNA:CBN ratio of the output material may be at least about 10.0:1.0.

In select embodiments of the present disclosure, the output material may further comprise THC, and the output material may have a CBNA:THC ratio of at least about 5.0:1.0. In select embodiments of the present disclosure, the CBNA:THC ratio of the output material may be at least about 10.0:1.0.

In select embodiments of the present disclosure, the output material may further comprises THCA, and the output material may have a CBNA:THCA ratio of at least about 5.0:1.0. In select embodiments of the present disclosure, the CBNA:THCA ratio of the output material may be at least about 10.0:1.0.

In select embodiments of the present disclosure, the benzoquinone reagent may comprise a compound as defined in formula (I) or formula (II):

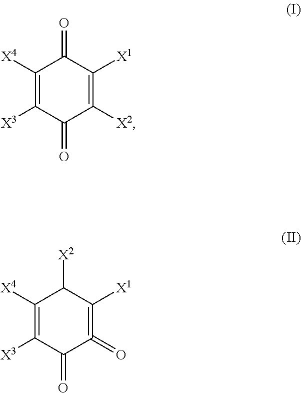

wherein X¹, X², X³, and X⁴ are each independently: H; a halide; a C_{<12}-hydrocarbyl; a C_{<12}-heteroaryl; a C_{<12}-heteroaralkyl; a C_{<12}-heteroaralkenyl; hydroxyl; a C_{<12}-alkoxy; a C_{<12}-amino; a C_{<12}-acyl; a C_{<12}-amide; a C_{<12}-ester; a C_{<12}-ketone; or a substituted analog thereof.

In select embodiments of the present disclosure, the benzoquinone reagent may comprise:

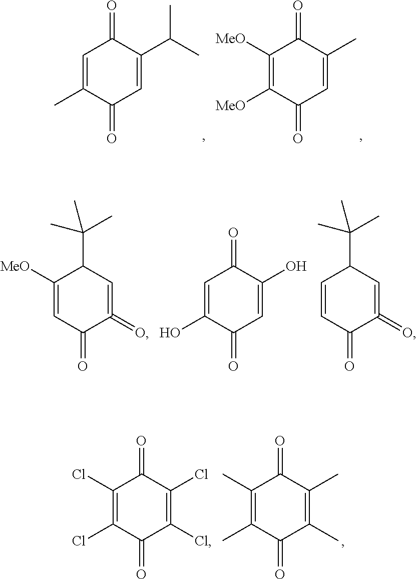

or a combination thereof.

In select embodiments of the present disclosure, the benzoquinone reagent may have an oxidation potential within the ranges set out in TABLE 1, which provides oxidation potentials for a series of benzoquinone reagents under non-limiting example conditions. Those skilled in the art who have benefited from the teachings of the present disclosure will readily understand the methods and standards required to determine the oxidation potential of any given benzoquinone reagent. Moreover, those skilled in the art who have benefited from the teaching of the present disclosure will recognize that the oxidation potential of any given benzoquinone reagent may be influenced by external factors such as solvent, pH, solute compositions, solute concentration, and the like.

TABLE 1
Oxidation potentials for a series of benzoquinone reagents under non-limiting example conditions.

| | | | | | E° | E° | E° | E° | E° |
|---|---|---|---|---|---|---|---|---|---|
| X₂ | X₃ | X₅ | X₆ | Σσ | [Q/Q⁻] | [Q⁻/Q²⁻] | [HQ/HQ⁻] | [Q, H⁺/HQ⁻] | [Q, 2H⁺/H₂Q] |
| H | H | H | H | 0.000 | 0.099 | 0.023 | 0.450 | 0.398 | 0.690 |
| C₆H₅ | H | H | H | −0.010 | 0.072 | 0.052 | 0.415 | 0.384 | 0.635 |
| CH₃ | H | H | H | −0.170 | 0.007 | −0.030 | 0.349 | 0.325 | 0.636 |
| C(CH₃)₃ | H | H | H | −0.200 | −0.041 | −0.096 | 0.320 | 0.294 | 0.602 |
| OCH₃ | H | H | H | −0.260 | −0.039 | −0.049 | 0.309 | 0.289 | 0.571 |
| N(CH₃)₂ | H | H | H | −0.830 | −0.221 | −0.144 | 0.124 | 0.182 | 0.466 |
| NH₂ | H | H | H | −0.660 | −0.193 | −0.117 | 0.042 | 0.175 | 0.456 |
| CH₂CH₃ | H | H | H | −0.150 | −0.025 | −0.068 | 0.321 | 0.300 | 0.605 |
| OH | H | H | H | −0.370 | 0.013 | −0.025 | 0.333 | 0.320 | 0.605 |
| OCH₂CH₃ | H | H | H | −0.280 | −0.070 | −0.069 | 0.300 | 0.271 | 0.541 |
| F | H | H | H | 0.340 | 0.231 | 0.153 | 0.559 | 0.467 | 0.687 |
| Cl | H | H | H | 0.370 | 0.242 | 0.195 | 0.595 | 0.491 | 0.706 |
| Br | H | H | H | 0.390 | 0.243 | 0.191 | 0.618 | 0.507 | 0.672 |
| SH | H | H | H | 0.150 | 0.110 | 0.086 | 0.436 | 0.403 | 0.665 |
| SiH₃ | H | H | H | 0.100 | 0.156 | 0.070 | 0.493 | 0.423 | 0.657 |
| CHO | H | H | H | 1.030 | 0.393 | 0.362 | 0.635 | 0.650 | 0.905 |
| COOCH₃ | H | H | H | 0.750 | 0.339 | 0.260 | 0.594 | 0.635 | 0.866 |
| CF₃ | H | H | H | 0.540 | 0.365 | 0.263 | 0.716 | 0.584 | 0.733 |
| CN | H | H | H | 1.000 | 0.479 | 0.401 | 0.853 | 0.686 | 0.778 |
| COOH | H | H | H | 0.770 | 0.592 | −0.068 | 0.621 | 0.644 | 0.799 |
| SO3— | H | H | H | 0.580 | 0.184 | 0.160 | 0.504 | 0.502 | 0.776 |
| NO2 | H | H | H | 1.270 | 0.613 | 0.688 | 1.007 | 0.833 | 0.938 |
| COCH₃ | H | H | H | 0.840 | 0.276 | 0.299 | 0.573 | 0.640 | 0.879 |
| C₆H₅ | C₆H₅ | H | H | −0.020 | 0.012 | 0.008 | 0.381 | 0.339 | 0.607 |
| CH₃ | CH₃ | H | H | −0.340 | −0.090 | −0.133 | 0.297 | 0.262 | 0.564 |
| C(CH₃)₃ | C(CH₃)₃ | H | H | −0.400 | −0.385 | −0.249 | 0.099 | 0.047 | 0.355 |
| OCH₃ | OCH₃ | H | H | −0.520 | −0.048 | 0.065 | 0.404 | 0.333 | 0.563 |
| N(CH₃)₂ | N(CH₃)₂ | H | H | −1.660 | −0.301 | −0.117 | 0.236 | 0.119 | 0.398 |
| NH₂ | NH₂ | H | H | −1.320 | −0.172 | −0.144 | 0.101 | 0.152 | 0.384 |
| CH₂CH₃ | CH₂CH₃ | H | H | −0.300 | −0.113 | −0.118 | 0.257 | 0.238 | 0.549 |
| OH | OH | H | H | −0.740 | 0.041 | 0.028 | 0.370 | 0.339 | 0.527 |
| OCH₂CH₃ | OCH₂CH₃ | H | H | −0.560 | −0.086 | 0.137 | 0.373 | 0.340 | 0.581 |
| F | F | H | H | 0.680 | 0.374 | 0.282 | 0.706 | 0.526 | 0.671 |
| Cl | Cl | H | H | 0.740 | 0.342 | 0.320 | 0.726 | 0.524 | 0.663 |
| Br | Br | H | H | 0.780 | 0.330 | 0.315 | 0.699 | 0.536 | 0.681 |
| SH | SH | H | H | 0.300 | 0.112 | 0.851 | 0.271 | 0.349 | 0.571 |
| SiH₃ | SiH₃ | H | H | 0.200 | 0.191 | 0.237 | 0.589 | 0.450 | 0.645 |
| CHO | CHO | H | H | 2.060 | 0.658 | 0.835 | 1.064 | 0.942 | 0.974 |
| COOCH₃ | COOCH₃ | H | H | 1.500 | 0.445 | 0.417 | 0.732 | 0.707 | 0.866 |
| CF₃ | CF₃ | H | H | 0.540 | 0.365 | 0.263 | 0.716 | 0.584 | 0.733 |
| CN | CN | H | H | 2.000 | 0.886 | 0.856 | 1.210 | 0.914 | 0.912 |
| COOH | COOH | H | H | 1.540 | 0.770 | 0.125 | 0.819 | 0.766 | 0.817 |
| SO3— | SO3— | H | H | 1.160 | 0.184 | 0.265 | 0.535 | 0.600 | 0.798 |
| NO2 | NO2 | H | H | 2.540 | 0.983 | 1.378 | 1.460 | 1.115 | 1.007 |
| COCH₃ | COCH₃ | H | H | 1.680 | 0.421 | 0.433 | 0.833 | 0.689 | 0.788 |
| C₆H₅ | H | C₆H₅ | H | −0.020 | 0.041 | 0.104 | 0.404 | 0.351 | 0.634 |
| CH₃ | H | CH₃ | H | −0.340 | −0.092 | −0.081 | 0.348 | 0.285 | 0.574 |
| C(CH₃)₃ | H | C(CH₃)₃ | H | −0.400 | −0.193 | −0.193 | 0.201 | 0.185 | 0.520 |
| OCH₃ | H | OCH₃ | H | −0.520 | −0.146 | −0.233 | 0.120 | 0.133 | 0.459 |
| N(CH₃)₂ | H | N(CH₃)₂ | H | −1.660 | −0.602 | −0.284 | −0.043 | −0.072 | 0.288 |
| NH₂ | H | NH₂ | H | −1.320 | −0.614 | −0.360 | −0.233 | −0.178 | 0.116 |
| CH₂CH₃ | H | CH₂CH₃ | H | −0.300 | −0.172 | −0.168 | 0.214 | 0.188 | 0.514 |
| OH | H | OH | H | −0.740 | −0.142 | −0.108 | 0.237 | 0.196 | 0.485 |
| OCH₂CH₃ | H | OCH₂CH₃ | H | −0.560 | −0.285 | −0.190 | 0.099 | 0.090 | 0.385 |
| F | H | F | H | 0.680 | 0.344 | 0.270 | 0.691 | 0.509 | 0.667 |
| Cl | H | Cl | H | 0.740 | 0.372 | 0.356 | 0.751 | 0.547 | 0.718 |
| Br | H | Br | H | 0.780 | 0.377 | 0.352 | 0.744 | 0.569 | 0.730 |
| SH | H | SH | H | 0.300 | 0.100 | 0.136 | 0.486 | 0.368 | 0.615 |
| SiH₃ | H | SiH₃ | H | 0.200 | 0.194 | 0.151 | 0.545 | 0.445 | 0.675 |
| CHO | H | CHO | H | 2.060 | 0.628 | 0.569 | 0.953 | 0.858 | 1.083 |
| COOCH₃ | H | COOCH₃ | H | 1.500 | 0.490 | 0.398 | 0.841 | 0.786 | 1.058 |
| CF₃ | H | CF₃ | H | 1.081 | 0.614 | 0.487 | 0.959 | 0.712 | 0.803 |
| CN | H | CN | H | 2.000 | 0.814 | 0.720 | 1.149 | 0.852 | 0.876 |
| COOH | H | COOH | H | 1.540 | 0.997 | −0.252 | 0.901 | 0.812 | 0.924 |
| SO3— | H | SO3— | H | 1.160 | 0.307 | 0.270 | 0.637 | 0.599 | 0.889 |
| NO2 | H | NO2 | H | 2.540 | 0.981 | 0.975 | 1.362 | 1.081 | 1.128 |
| COCH₃ | H | COCH₃ | H | 1.680 | 0.463 | 0.363 | 0.718 | 0.739 | 1.076 |
| C₆H₅ | H | H | C₆H₅ | −0.020 | 0.019 | 0.070 | 0.364 | 0.345 | 0.599 |
| CH₃ | H | H | CH₃ | −0.340 | −0.088 | −0.095 | 0.241 | 0.258 | 0.553 |
| C(CH₃)₃ | H | H | C(CH₃)₃ | −0.400 | −0.192 | −0.274 | 0.124 | 0.157 | 0.467 |
| OCH₃ | H | H | OCH₃ | −0.520 | −0.154 | −0.123 | 0.148 | 0.215 | 0.493 |
| N(CH₃)₂ | H | H | N(CH₃)₂ | −1.660 | −0.468 | −0.255 | −0.017 | 0.037 | 0.338 |
| NH₂ | H | H | NH₂ | −1.320 | −0.345 | −0.265 | −0.143 | 0.020 | 0.285 |
| CH₂CH₃ | H | H | CH₂CH₃ | −0.300 | −0.142 | −0.143 | 0.199 | 0.204 | 0.506 |
| OH | H | H | OH | −0.740 | −0.034 | −0.060 | 0.263 | 0.269 | 0.518 |
| OCH₂CH₃ | H | H | OCH₂CH₃ | −0.560 | −0.173 | −0.167 | 0.164 | 0.175 | 0.438 |
| F | H | H | F | 0.680 | 0.382 | 0.286 | 0.679 | 0.551 | 0.675 |
| Cl | H | H | Cl | 0.740 | 0.389 | 0.350 | 0.745 | 0.584 | 0.683 |
| Br | H | H | Br | 0.780 | 0.387 | 0.358 | 0.776 | 0.616 | 0.734 |
| SH | H | H | SH | 0.030 | 0.135 | 0.149 | 0.439 | 0.402 | 0.548 |
| SiH₃ | H | H | SiH₃ | 0.200 | 0.203 | 0.148 | 0.569 | 0.474 | 0.615 |
| CHO | H | H | CHO | 2.060 | 0.634 | 0.673 | 0.990 | 0.880 | 1.021 |
| COOCH₃ | H | H | COOCH₃ | 1.500 | 0.518 | 0.437 | 0.775 | 0.740 | 0.939 |
| CF₃ | H | H | CF₃ | 1.080 | 0.620 | 0.496 | 1.025 | 0.785 | 0.797 |
| CN | H | H | CN | 2.000 | 0.815 | 0.734 | 1.285 | 0.970 | 0.874 |
| COOH | H | H | COOH | 1.540 | 0.988 | −0.106 | 0.809 | 0.788 | 0.847 |
| SO3— | H | H | SO3— | 1.160 | 0.302 | 0.269 | 0.614 | 0.574 | 0.810 |
| NO2 | H | H | NO2 | 2.540 | 0.944 | 1.081 | 1.488 | 1.102 | 1.047 |
| COCH₃ | H | H | COCH₃ | 1.680 | 0.375 | 0.513 | 0.740 | 0.720 | 0.926 |
| C₆H₅ | C₆H₅ | C₆H₅ | H | −0.030 | −0.024 | 0.014 | 0.334 | 0.324 | 0.588 |
| CH₃ | CH₃ | CH₃ | H | −0.510 | −0.211 | −0.192 | 0.162 | 0.158 | 0.485 |
| C(CH₃)₃ | C(CH₃)₃ | C(CH₃)₃ | H | −0.600 | −0.560 | −0.468 | −0.088 | −0.079 | 0.229 |
| OCH₃ | OCH₃ | OCH₃ | H | −0.780 | −0.213 | −0.010 | 0.233 | 0.213 | 0.455 |
| N(CH₃)₂ | N(CH₃)₂ | N(CH₃)₂ | H | −2.490 | −0.699 | −0.262 | −0.136 | −0.027 | 0.370 |
| NH₂ | NH₂ | NH₂ | H | −1.980 | −0.556 | −0.361 | −0.163 | −0.129 | 0.120 |
| CH₂CH₃ | CH₂CH₃ | CH₂CH₃ | H | −0.450 | −0.223 | −0.205 | 0.125 | 0.154 | 0.491 |
| OH | OH | OH | H | −1.110 | −0.079 | −0.030 | 0.246 | 0.235 | 0.444 |
| OCH₂CH₃ | OCH₂CH₃ | OCH₂CH₃ | H | −0.840 | −0.290 | 0.048 | 0.236 | 0.205 | 0.465 |
| F | F | F | H | 1.110 | 0.499 | 0.405 | 0.824 | 0.606 | 0.691 |
| Cl | Cl | Cl | H | 1.170 | 0.472 | 0.472 | 0.877 | 0.626 | 0.698 |
| Br | Br | Br | H | 0.450 | 0.462 | 0.477 | 0.848 | 0.643 | 0.720 |
| SH | SH | SH | H | 0.450 | 0.117 | 0.217 | 0.511 | 0.407 | 0.491 |
| SiH₃ | SiH₃ | SiH₃ | H | 0.300 | 0.233 | 0.272 | 0.611 | 0.475 | 0.611 |
| CHO | CHO | CHO | H | 3.090 | 0.796 | 0.978 | 1.257 | 1.072 | 1.167 |
| COOCH₃ | COOCH₃ | COOCH₃ | H | 2.250 | 0.586 | 0.559 | 0.938 | 0.849 | 1.053 |
| CF₃ | CF₃ | CF₃ | H | 1.620 | 0.845 | 0.748 | 1.292 | 0.918 | 0.875 |
| CN | CN | CN | H | 3.000 | 1.178 | 1.122 | 1.553 | 1.134 | 0.968 |
| COOH | COOH | COOH | H | 2.310 | 1.149 | −0.065 | 1.060 | 0.929 | 0.966 |
| SO3— | SO3— | SO3— | H | 1.740 | 0.256 | 0.353 | 0.646 | 0.665 | 0.902 |
| NO2 | NO2 | NO2 | H | 3.810 | 1.261 | 1.510 | 1.701 | 1.269 | 1.147 |
| COCH₃ | COCH₃ | COCH₃ | H | 2.520 | 0.557 | 0.518 | 0.935 | 0.865 | 0.898 |
| C₆H₅ | C₆H₅ | C₆H₅ | C₆H₅ | −0.040 | −0.084 | 0.009 | 0.367 | 0.281 | 0.561 |
| CH₃ | CH₃ | CH₃ | CH₃ | −0.040 | −0.084 | 0.009 | 0.367 | 0.281 | 0.561 |
| C(CH₃)₃ | C(CH₃)₃ | C(CH₃)₃ | C(CH₃)₃ | −0.800 | −1.107 | −0.804 | −0.388 | −0.509 | −0.153 |
| OCH₃ | OCH₃ | OCH₃ | OCH₃ | −1.040 | −0.229 | 0.111 | 0.370 | 0.220 | 0.465 |
| N(CH₃)₂ | N(CH₃)₂ | N(CH₃)₂ | N(CH₃)₂ | −3.320 | −0.629 | −0.322 | −0.253 | −0.138 | 0.203 |
| NH₂ | NH₂ | NH₂ | NH₂ | −2.640 | −0.571 | −0.456 | −0.197 | −0.192 | 0.028 |
| CH₂CH₃ | CH₂CH₃ | CH₂CH₃ | CH₂CH₃ | −0.600 | −0.372 | −0.347 | 0.066 | 0.032 | 0.384 |
| OH | OH | OH | OH | −1.480 | −0.077 | −0.039 | 0.295 | 0.183 | 0.379 |
| OCH₂CH₃ | OCH₂CH₃ | OCH₂CH₃ | OCH₂CH₃ | −1.120 | −0.305 | 0.238 | 0.388 | 0.290 | 0.527 |
| F | F | F | F | 1.360 | 0.638 | 0.531 | 0.986 | 0.670 | 0.731 |
| Cl | Cl | Cl | Cl | 1.480 | 0.564 | 0.588 | 1.003 | 0.663 | 0.684 |
| Br | Br | Br | Br | 1.560 | 0.539 | 0.581 | 0.960 | 0.660 | 0.720 |
| SH | SH | SH | SH | 0.600 | 0.111 | 0.279 | 0.526 | 0.342 | 0.453 |
| SiH₃ | SiH₃ | SiH₃ | SiH₃ | 0.400 | 0.247 | 0.322 | 0.675 | 0.459 | 0.558 |
| CHO | CHO | CHO | CHO | 4.120 | 0.873 | 1.005 | 1.319 | 1.099 | 1.221 |
| COOCH₃ | COOCH₃ | COOCH₃ | COOCH₃ | 3.000 | 0.744 | 0.680 | 1.064 | 0.909 | 1.052 |
| CF₃ | CF₃ | CF₃ | CF₃ | 2.160 | 0.972 | 0.902 | 1.397 | 0.937 | 0.833 |
| CN | CN | CN | CN | 4.000 | 1.48 | 1.430 | 1.832 | 1.271 | 1.025 |
| COOH | COOH | COOH | COOH | 3.080 | 1.278 | 0.068 | 1.143 | 0.970 | 0.980 |
| SO3— | SO3— | SO3— | SO3— | 2.320 | 0.084 | 0.348 | 0.613 | 0.546 | 0.846 |
| NO2 | NO2 | NO2 | NO2 | 5.080 | 1.613 | 1.662 | 1.939 | 1.441 | 1.231 |
| COCH₃ | COCH₃ | COCH₃ | COCH₃ | 3.360 | 0.663 | 0.657 | 0.914 | 0.768 | 0.865 |
| CN | CN | Cl | Cl | 2.740 | 1.096 | 1.079 | 1.461 | 1.027 | 0.884 |

In select embodiments of the present disclosure, the contacting of the input material with the benzoquinone reagent may comprise introducing the benzoquinone reagent to the input material at a benzoquinone:THCA ratio of between about 1.0:1.0 and about 10.0:1.0 on a molar basis. In select embodiments of the present disclosure, the benzoquinone:THCA ratio may be between about 2.0:1.0 and about 4.0:1.0 on a molar basis. In a particular embodiment, the benzoquinone:THCA ratio is about 2.5:1.0, about 2.6:1.0, about 2.7:1.0, about 2.8:1.0, about 2.9:1.0, about 3.0:1.0, about 3.1:1.0, about 3.2:1.0, about 3.3:1.0, about 3.4:1.0, or about 3.5:1.0 on a molar basis.

In select embodiments of the present disclosure, the benzoquinone reagent (both spent and unreacted) may be separated from the crude product mixture and reactivated such that it may be reused in a further reaction. Those skilled in the art who have benefitted from the teachings of the present disclosure will recognize suitable methods for regenerating the benzoquinone reagent such as treatment with a strong reductant.

In select embodiments of the present disclosure, the target reaction-temperature range may be between about 20° C. and about 190° C. In select embodiments of the present disclosure, the target reaction-temperature range may be between about 60° C. and about 130° C. In a particular embodiment, the target reaction temperature is about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., or about 105° C. In another particular embodiment, the target temperature is about 50° C., about 51° C., about 52° C., about 53° C., about 54° C., about 55° C., about 56° C., about 57° C., about 58° C., about 59° C., or about 60° C. Those skilled in the art who have benefitted from the teachings of the present disclosure will recognize that selecting a target-reaction temperature range may be done having regard to the particulars of the input material, the desired extent of upgrading, the particulars of the benzoquinone reagent, the particulars of the solvent system (or lack thereof), the reaction time, and the like.

In select embodiments of the present disclosure, the target reaction-time range may be between about 1 h and about 100 h. In select embodiments of the present disclosure, the target reaction-temperature range may be between about 20 h and about 80 h. In a particular embodiment, the reaction time is about 7 h, about 8 h, about 9 h, about 10 h, about 11 h, about 12 h, about 13 h, about 14 h, or about 15 h. In another particular embodiment, the reaction time is about 18 h, about 20 h, about 22 h, about 24 h, about 26 h, about 28 h, or about 30 h. In a further particular embodiment, the reaction time is about 68 h, about 70 h, about 72 h, about 74 h, or about 76 h. Those skilled in the art who have benefitted from the teachings of the present disclosure will recognize that selecting a target-reaction time range may be done having regard to the particulars of the input material, the desired extent of upgrading, the particulars of the benzoquinone reagent, the particulars of the solvent system (or lack thereof), the reaction temperature, and the like.

In select embodiments of the present disclosure, the contacting of the input material with the benzoquinone reagent is in the presence of solvent. In instances where a solvent is employed, the solvent may be protic or aprotic. By way of non-limiting example, an aprotic-solvent system may comprise dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or a combination thereof. As will be appreciated by those skilled in the art who have benefitted from the present disclosure, aprotic solvent systems may comprise small amounts of protic species, the quantities of which may be influenced by the extent to which drying and/or degassing procedures are employed. By way of non-limiting example a protic-solvent system may comprise methanol, ethanol, 1-propanol, 2-propanol, 1-butanol, 2-butanol, water, acetic acid, formic acid, 3-methyl-1-butanol, 2-methyl-1-propanol, 1-pentanol, nitromethane, or a combination thereof. In select embodiments of the present disclosure, the solvent is pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

The CBNA provided by the methods disclosed herein may be decarboxylated by methods known in the art to provide CBN. Thus, select embodiments of the present disclosure relate to methods of converting THCA to CBN.

### EXAMPLES

The following examples describe a series of experiments in which THCA was contacted with a benzoquinone reagent to provide CBNA as generally characterized in non-limiting SCHEME 1.

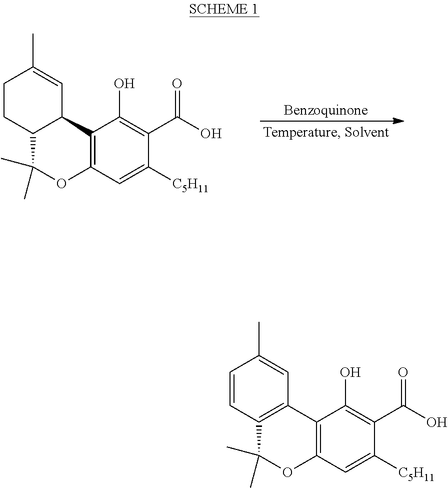

### Example 1

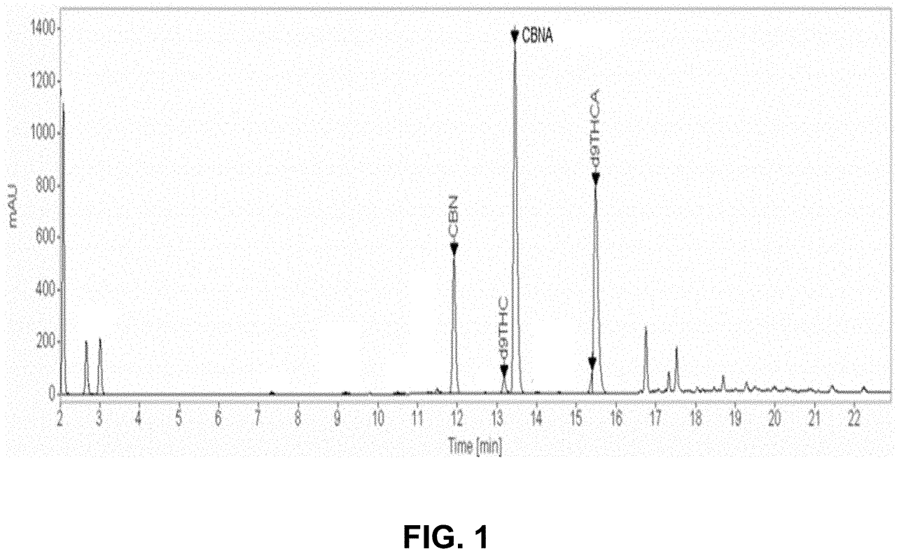

A mixture of crystalline THCA (0.10 g, [>99 wt. % 4⁹-THCA], 0.28 mmol), heptane (10 mL), and tetrachloro-1,4-benzoquinone (0.27 g, 1.10 mmol) was stirred and heated to 100° C. for 24 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with heptane, filtered using a Buchner funnel equipped with a glass frit, and concentrated in vacuo to provide 0.14 g of output material. The output material was analyzed by HPLC-DAD to provide the chromatogram set out in FIG. 1, which shows CBNA as a primary product, CBN as a secondary product, THC as a tertiary product, and a substantial amount of unreacted THCA.

### Example 2

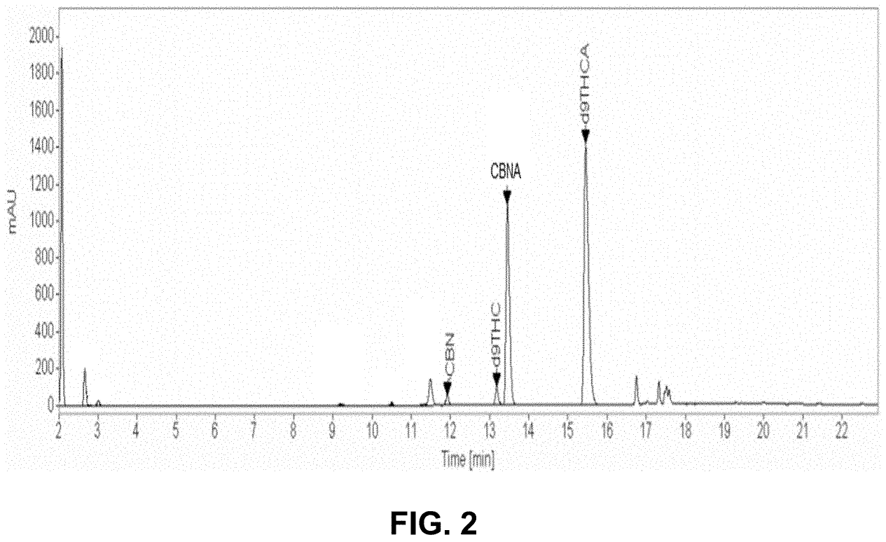

A mixture of crystalline THCA (0.10 g, [>99 wt. % 4⁹-THCA], 0.28 mmol), ethyl acetate (10 mL), and tetrachloro-1,4-benzoquinone (0.27 g, 1.10 mmol) was stirred and heated to 85° C. for 24 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with pentane, filtered using a Buchner funnel equipped with a glass frit, and concentrated in vacuo to provide 0.15 g of output material. The output material was analyzed by HPLC-DAD to provide the chromatogram shown in FIG. 2, which shows CBNA as a primary product, small amounts of CBN and THC, and a substantial amount of unreacted THCA. The chromatogram of FIG. 2 indicates a high CBNA:CBN ratio (and a high CBNA:THC ratio) in the output material, which suggests the method induces oxidation preferentially over decarboxylation.

### Example 3

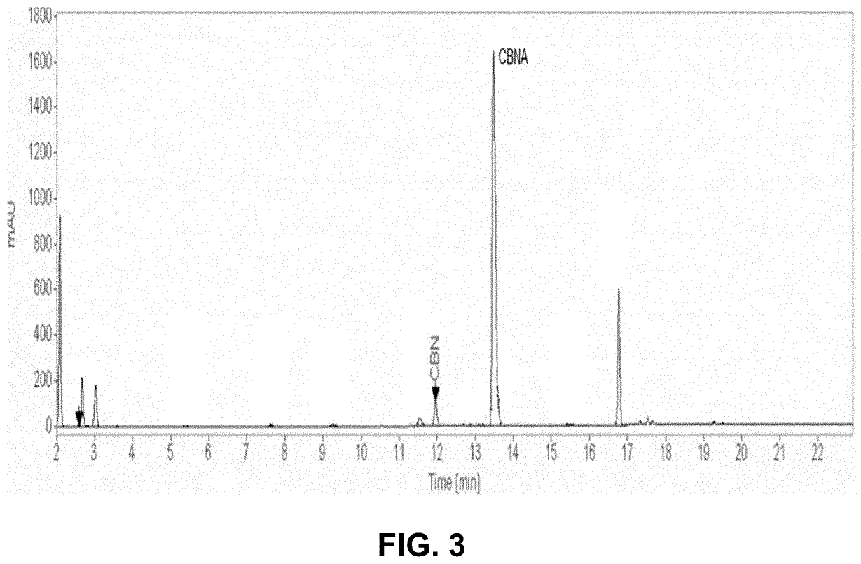

A mixture of crystalline THCA (0.10 g, [>99 wt. % Δ⁹-THCA], 0.28 mmol), ethyl acetate (10 mL), and tetrachloro-1,4-benzoquinone (0.27 g, 1.10 mmol) was stirred and heated to 85° C. for 72 hours to form a crude product mixture. The crude product mixture was cooled to ambient temperature and filtered using a Buchner funnel equipped with a glass frit to separate suspended solids from a filtrate. The filtrate was concentrated in vacuo to provide a crude product residue that was triturated with pentane, filtered using a Buchner funnel equipped with a glass frit, and concentrated in vacuo to provide 0.13 g of output material. The output material was analyzed by HPLC-DAD to provide the chromatogram shown in FIG. 3, which shows CBNA as a primary product, a small amount of CBN, and an unknown by-product eluting at 16.77 min. The chromatogram of FIG. 3 indicates a high CBNA:CBN ratio, no observable THC formation, and no unreacted THCA in the output material.

### Example 4

A THCA-rich resin (5 g; 60 w/w % THCA, 0% CBN, 0% CBNA) and 3 equivalents of chloranil were suspended in heptane and heated at 55° C. for about 72 h. The crude product mixture was cooled to ambient temperature, filtered, and the solvent was removed under vacuum. Analysis of the crude product material by HPLC-DAD showed the material comprised 40 w/w % THCA, 15 w/w % CBNA, and 3 w/w % CBN. The crude product material was purified by column chromatography using a silica column and heptane: TBME.

### Example 5

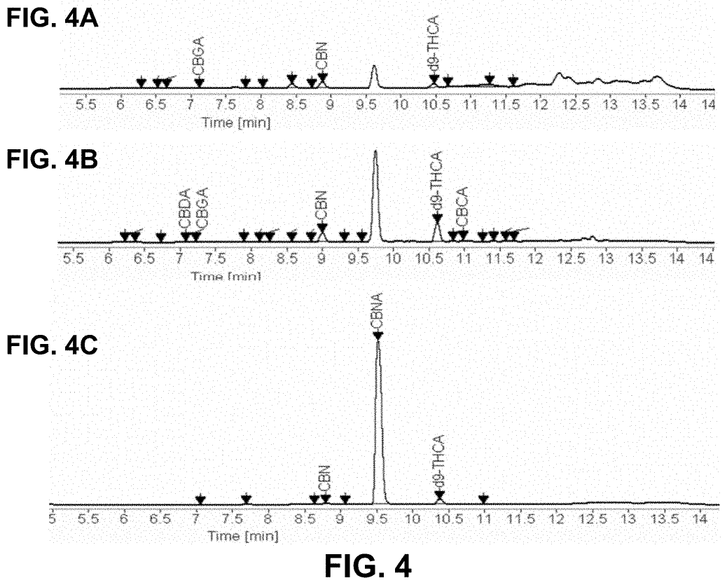

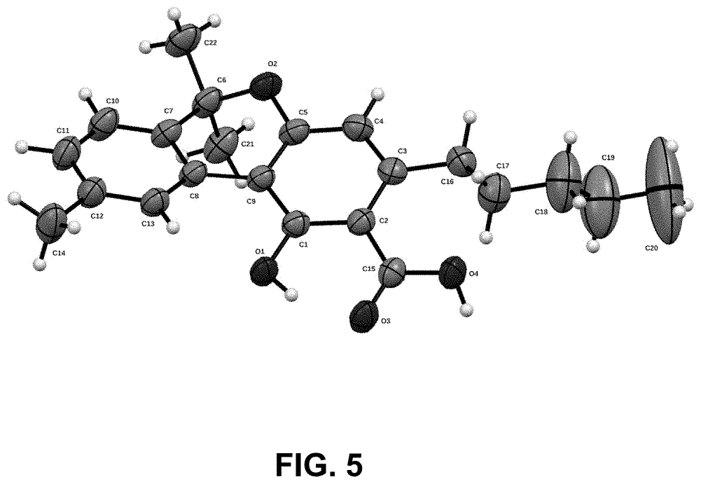

A THCA-rich resin (5 g; 60 w/w % THCA, 0% CBN, 0% CBNA) and 3 equivalents of chloranil were suspended in heptane and heated at 85° C. for about 9 h. The crude product mixture was cooled to ambient temperature, filtered, and the solvent was removed under vacuum. Analysis of the crude product material by HPLC-DAD showed the material comprised 6 w/w % THCA, 26 w/w % CBNA and 5 w/w % CBN (FIG. 4A). A first round of purification provided a product mixture comprising about 55% CBNA (FIG. 4B). A second round of purification provided Colourless block-shaped crystals of CBNA (90-99% purity by HPLC DAD; FIG. 4C) suitable for single-crystal X-ray diffraction (FIG. 5).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including ” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A method for converting tetrahydrocannabinolic acid (THCA) to cannabinolic acid (CBNA), the method comprising contacting an input material comprising the THCA with a benzoquinone reagent.

2. (canceled)

3. The method of claim 1, wherein the input material is derived from marijuana biomass.

4. The method of claim 1, wherein the input material is a cannabis distillate, a cannabis resin, a cannabis extract, or a combination thereof.

5. The method of claim 1, wherein the input material is THCA in an isolated form.

6. The method of claim 1, wherein at least about 60 weight % of the THCA is Δ⁹-THCA.

7. The method of claim 6, wherein at least about 95 weight % of the THCA is Δ⁹-THCA.

8. (canceled)

14. The method of claim 1, wherein the benzoquinone reagent comprises a compound as defined in formula (I) or formula (II): 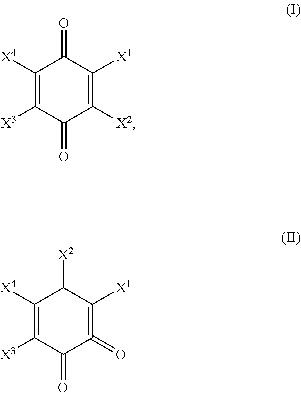
wherein X¹, X², X³, and X⁴ are each independently: H; a halide; a C_{<12}-hydrocarbyl; a C_{<12}-heteroaryl; a C_{<12}-heteroaralkyl; a C_{<12}-heteroaralkenyl; hydroxyl; a C_{<12}-alkoxy; a C_{<12}-amino; a C_{<12}-acyl; a C_{<12}-amide; a C_{<12}-ester; a C_{<12}-ketone; or a substituted analog thereof.

15. The method of claim 1, wherein the benzoquinone reagent comprises: 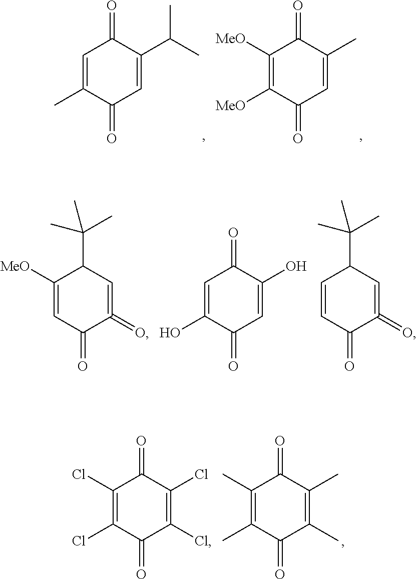
or a combination thereof.

16. (canceled)

17. (canceled)

18. The method of claim 1, wherein the contacting of the input material with the benzoquinone reagent is at a benzoquinone:THCA ratio of between about 1.0:1.0 and about 10.0:1.0 on a molar basis.

19. The method of claim 18, wherein the benzoquinone:THCA ratio is between about 2.0:1.0 and about 4.0:1.0 on a molar basis.

20. The method of claim 1, wherein the contacting of the input material with the benzoquinone reagent is at a temperature of between about 20° C. and about 190° C.

21. The method of claim 1, wherein the contacting of the input material with the benzoquinone reagent is at a temperature of between about 80° C. and about 110° C.

22. (canceled)

23. (canceled)

24. The method of claim 1, wherein the contacting of the input material with the benzoquinone reagent is in the presence of a solvent.

25. The method of claim 24, wherein the solvent is pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

26. A method for converting tetrahydrocannabinolic acid (THCA) to cannabinolic acid (CBNA), the method comprising contacting the THCA with tetrachloro-1,4-benzoquinone at a temperature of between about 60° C. and about 130° C.

27. The method of claim 14, wherein the benzoquinone reagent comprises:
a compound as defined in formula (I) where X¹═H, X²═H, X³═H, and X⁴═H,
a compound as defined in formula (I) where X¹═CN, X²═CN, X³═Cl, and X⁴═Cl,
a compound as defined in formula (II) where X¹═H, X²═C(CH₃)₃, X³═C(CH₃)₃, and x⁴═H,
a compound as defined in formula (II) where X¹═Cl, X²═Cl, X³═Cl, and X⁴═Cl,
a compound as defined in formula (I) where X¹═Cl, X²═Cl, X³═Cl, and X⁴═Cl,
a compound as defined in formula (II) where X¹═H, X²═C(CH₃)₃, X³═H, and X⁴═H,
a compound as defined in formula (I) where X¹═H, X^{2═}OH, X³═H, and X⁴═H,
a compound as defined in formula (II) where X¹═H, X²═C(CH₃)₃, X³═H, and X⁴═OCH₃, or
a compound as defined in formula (II) where X¹═H, X²═H, X³═H, and X⁴═OCH₃.

28. The method of claim 24, wherein the solvent is a protic solvent.

29. The method of claim 24, wherein the solvent is an aprotic solvent.
